(19) United States
(12) Patent Application Publication
(10) Pub. No.: US 2010/0102253 A1
(43) Pub. Date: Apr. 29, 2010

(54) STERILIZATION APPARATUS FOR FABRIC AND SHOES

(76) Inventors: KUO-JEN CHANG, Taoyuan (TW); Kun-Chia Wu, Dali City (TW)

Correspondence Address: Dr. BANGER SHIA;Patent Office of Bang Shia, 102 Lindencrest Ct, Sugar Land, TX, 77479-5201, US

(21) Appl. No.: 12/563,914

(22) Filed: Sep. 21, 2009

(30) Foreign Application Priority Data
Oct. 28, 2008 (TW) 097219240

(51) Int. Cl. A61L 2/08; A61L 2/10

(52) U.S. Cl. 250/492.1

(57) ABSTRACT

The invention discloses a sterilization apparatus for a fabric and shoes. The sterilization apparatus of the invention includes an organization member for containing the fabric or shoes. The sterilization apparatus of the invention further includes a light sterilization type member and/or an exsiccating member disposed with the organization member for sterilizing or drying the fabric or shoes.

### BACKGROUND OF THE INVENTION

1. Field of the Invention

The present invention relates generally to a sterilization apparatus for fabric and shoes, and more particularly, to a sterilization apparatus including a light sterilization type member and/or an exsiccating member for sterilizing or drying the fabric or shoes.

2. Description of the Prior Art

Today's wire coat hanger was inspired by a clothes hook patented in 1869, by O. A. North. Albert J. Parkhouse, an employee of Timberlake Wire and Novelty Company in Jackson, created a coat hanger in 1903 and granted a patent about the coat hanger, in response to co-workers' complaints of too few coat hooks.

For example, TW Pat. No. M321265 entitled “COAT HANGER” disclosed a quadrangular coat hanger which includes a main body and a rotating body. The main body has a hanging portion. At least one pivot base, having at least a hole and a bump, is disposed on each sides of the main body. The bump is disposed on the outside of the pivot base and adjacent to the hole. The rotating body is disposed through the pivot base respectively, and the rotating body has a sleeve. A mounting hole is disposed on one end of the sleeve, and a shaft is disposed on the other end of the sleeve. The elongating end of the shaft is inserted in the hole. A first against end and a second against end are disposed at the joint portion of the sleeve and the shaft for selectively against a connecting member with the bump. The connecting member has at least two vertical portions and at least one horizontal portion. The first ends of the vertical portions are connected with the ends of the horizontal portion respectively, and the second ends of the vertical portions are inserted in the mounting hole and against to the bump with the first against end and the second against end. Therefore, the vertical portions can moves between a first position and a second position relative to the main body.

Moreover, TW Pat. No. M321038 entitled “FOLDABLE COAT HANGER” disclosed an upright coat hanger which includes two main shafts, two up shafts, two upright shafts and a certain of horizontal shafts. Wherein, the main shafts are long tubes and two triangular mounting blocks are disposed opposite to each other on the outside of each of the middle of the main shafts. Accordingly, each of the main shafts is separated by the mounting blocks, and the upper part thereof can be used as a fixing part and the lower part thereof can be used as a supporting part. Moreover, a recessive part with same shape and smaller size of the mounting blocks is formed on each of the mounting blocks. An inclined blocking edge is formed on the side of the recessive part away from the main shaft, and a pivot hole is formed on each side of the recessive part. Moreover, the diameters of the up shafts and the upright shafts are slightly smaller than the width of the recessive part, so that the up shafts and the upright shafts can be inserted into the recessive parts of the upper part and the lower parts of the main shafts respectively. After that, a connecting member can be inserted into the pivot holes and holes on the up shafts and the upright shafts to mount the up shafts and the upright shafts on the mounting blocks. Additionally, the horizontal shafts are disposed between the mounting part of the main shafts and the top of the up shafts, so that the main shafts can be connected with each other by the horizontal shafts.

To sum up, the design of conventional coat hangers focus on different structures for organization or on skid-proof structure. However, the conventional coat hangers can not be used for inhibiting the growth of bacteria or fungus.

### SUMMARY OF THE INVENTION

Accordingly, a scope of the present invention is to provide a sterilization apparatus for a fabric and shoes. The sterilization apparatus of the invention uses a light sterilization type member for sterilizing the fabric and shoes.

Another scope of the present invention is to provide a sterilization apparatus for a fabric and shoes. The sterilization apparatus of the invention uses an exsiccating member for drying the fabric and shoes.

To achieve the above-mentioned objects and efficiencies, the sterilization apparatus includes an organization member, such as a wardrobe, a coat hanger or a shoes cabinet, for containing the fabric, such as clothes or table clothes. Particularly, the sterilization apparatus of the invention includes a light sterilization type member and/or an exsiccating member disposed with the organization member for sterilizing or drying the fabric or shoes.

The objective of the present invention will no doubt become obvious to those of ordinary skill in the art after reading the following detailed description of the preferred embodiment, which is illustrated in the various figures and drawings.

### BRIEF DESCRIPTION OF THE APPENDED DRAWINGS

FIG. 1 illustrates the structure of a sterilization apparatus with a sterilization member of a preferred embodiment of the invention.

FIG. 2 illustrates the structure of a sterilization apparatus with a sterilization member of another preferred embodiment of the invention.

FIG. 3 illustrates the structure of a sterilization apparatus with an exsiccating member of another preferred embodiment of the invention.

FIG. 4 illustrates the structure of a sterilization apparatus with an exsiccating member of another preferred embodiment of the invention.

FIG. 5 illustrates the structure of a sterilization apparatus with a sterilization member and an exsiccating member of another preferred embodiment of the invention.

FIG. 6 illustrates the structure of a sterilization apparatus with a sterilization member, an exsiccating member and a ventilating member of another preferred embodiment of the invention.

FIG. 7 illustrates the structure of a sterilization apparatus with a sterilization member of an embodiment of the invention.

FIG. 8 illustrates the structure of a sterilization apparatus with a sterilization member of an embodiment of the invention.

### DETAILED DESCRIPTION OF THE INVENTION

In the prior art, the organization member, such as a wardrobe, a coat hanger, a shoes cabinet or other organization members, is used for containing or hanging fabrics and/or shoes. However, the conventional coat hangers can not be used for inhibiting the growth of bacteria or fungus. Accordingly, the sterilization apparatus of the invention can be used for solving said problem.

The sterilization apparatus of the invention can be used for organizing fabrics and shoes. The sterilization apparatus includes an organization member, such as a wardrobe, a coat hanger or a shoes cabinet. Moreover, the sterilization apparatus further includes a light sterilization type member disposed with the organization member.

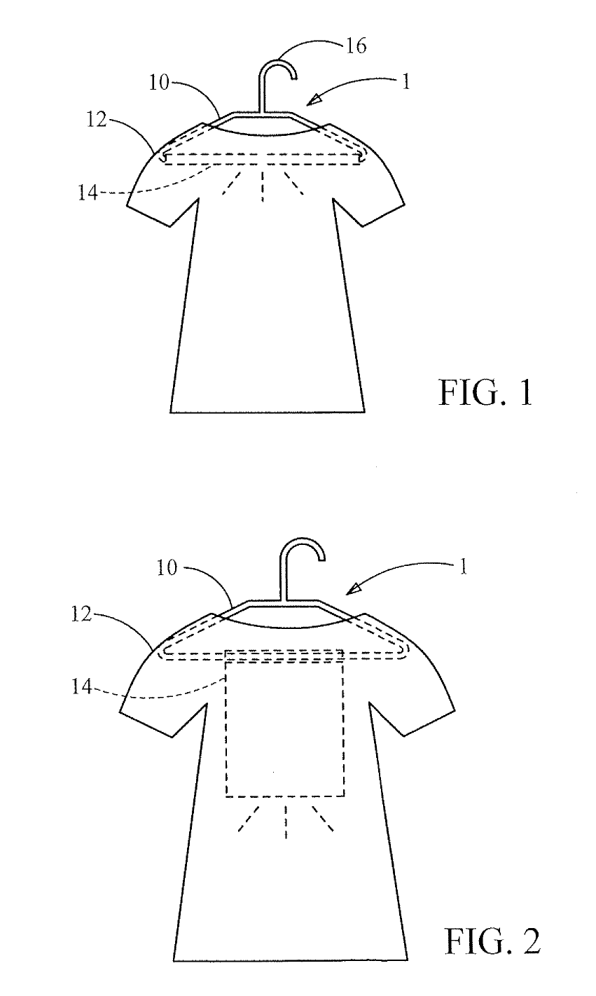

Please refer to FIG. 1, which illustrates the structure of a sterilization apparatus with a sterilization member of a preferred embodiment of the invention. As shown in FIG. 1, the sterilization apparatus 1 of the invention includes the coat hanger 10 and the light sterilization type member 14. The coat hanger 10 is triangular for hanging a fabric 12. In practice, the coat hanger 10 can be quadrangular or polygonal, and it can be an upright type. The light sterilization type member 14 is disposed in the coat hanger 10 and connected with the coat hanger 10. Moreover, when the fabric 12 is hanged on the coat hanger 10, the light sterilization type member 14 is inside the fabric 12. A hook 16 is disposed at the top of the coat hanger 10 for hooking the shaft in a closet or an outdoor clotheshorse. In practice, the fabric 12 can be a weave product or a non-weave product.

In practice, the light sterilization type member 14 can be an ultraviolet fluorescent light or an ultraviolet light-emitting diode. Practically, a photocatalyst layer can be coated on the surface of the ultraviolet fluorescent light, the ultraviolet light-emitting diode or the fluorescent lamp. Because the ultraviolet fluorescent light has wave lengths such as UV-A, UV-B and UV-C which belong to invisible light and can be used to sterilize objected by illuminating thereon, it can be disposed in the coat hanger 10. Moreover, the photocatalyst can also be activated by the ultraviolet fluorescent light or the ultraviolet light-emitting diode to sterilize the air. In practice, some photocatalysts can be activated by visible light. Accordingly, the light source can also be a fluorescent lamp or a tungsten lamp. Moreover, in practice, the coat hanger 10 can be replaced by a closet.

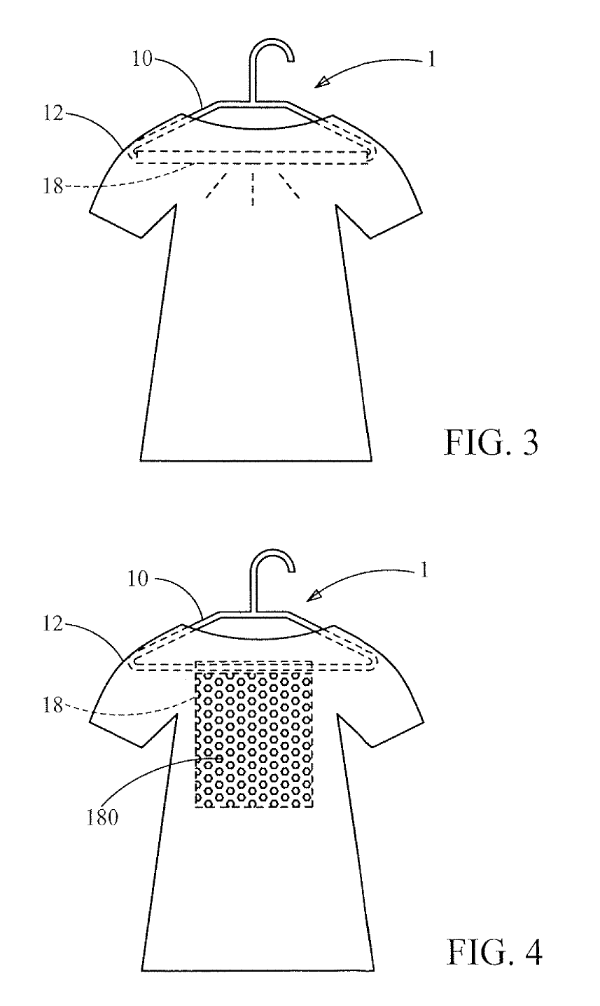

Please refer to FIG. 2, which illustrates the structure of a sterilization apparatus with a sterilization member of another preferred embodiment of the invention. As shown in FIG. 2, the light sterilization type member 14 is disposed under the coat hanger 10 in the embodiment. In practice, because the length of different fabrics 12 may be different, the length of the light sterilization type member 14 can be adjusted relatively.

Please refer to FIG. 3, which illustrates the structure of a sterilization apparatus with an exsiccating member of another preferred embodiment of the invention. As shown in FIG. 3, the sterilization apparatus 1 in the embodiment includes a coat hanger 10 and an exsiccating member 18. The coat hanger 10 is used for hanging a fabric 12. Moreover, the exsiccating member 18 can be disposed in the coat hanger 10 or under the coat hanger 10 and connected with the coat hanger 10 for drying the fabric 12. In practice, the exsiccating member 18 can be an infrared light, a heating member or an electronic exsiccating member.

Please refer to FIG. 4, which illustrates the structure of a sterilization apparatus with an exsiccating member of another preferred embodiment of the invention. As shown in FIG. 4, the exsiccating member 18 contains exsiccating materials or heating materials 180 for drying the fabric 12 or the air.

Please refer to FIG. 5, which illustrates the structure of a sterilization apparatus with a sterilization member and an exsiccating member of another preferred embodiment of the invention. As shown in FIG. 5, the sterilization apparatus 1 in the embodiment includes both of the light sterilization type member 14 and the exsiccating member 18 disposed under the coat hanger 10 to sterilize and dry the fabric 12 at the same time.

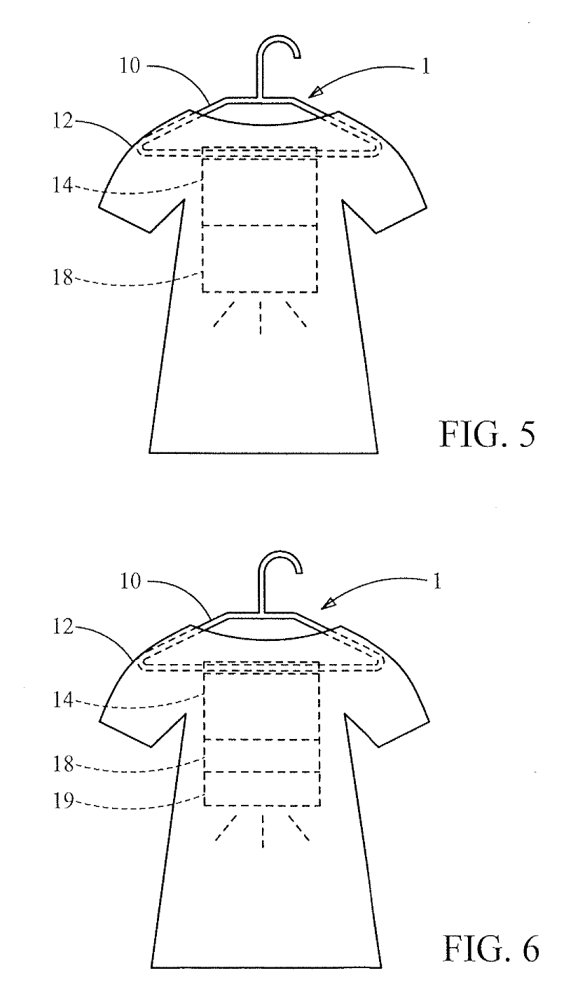

Please refer to FIG. 6, which illustrates the structure of a sterilization apparatus with a sterilization member, an exsiccating member and a ventilating member of another preferred embodiment of the invention. As shown in FIG. 6, the sterilization apparatus 1 of the embodiment further includes a ventilating member 19, such as a fan, for enhancing air flow and advancing sterilization and exsiccation effect. Moreover, the ventilating member 19 can be applied in the other embodiments as mentioned above.

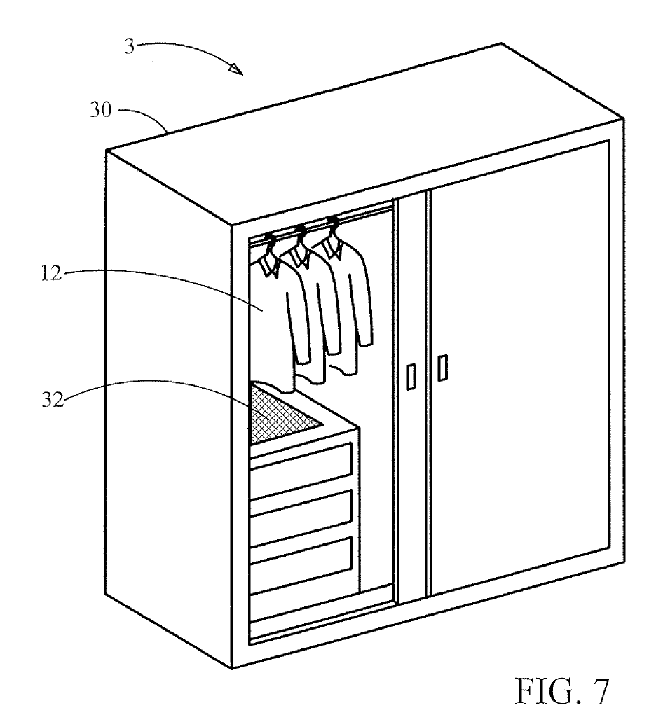

Please refer to FIG. 7, which illustrates the structure of a sterilization apparatus with a sterilization member of an embodiment of the invention. The sterilization apparatus 3 of the embodiment includes a cabinet 30 for hanging clothes 12. Moreover, in the embodiment, the sterilization apparatus 3 further includes a sterilization member 32, such as light sterilization type member. In practice, the sterilization apparatus 3 can further includes the above-mentioned exsiccating member and ventilating member. The functionalities and types of said members are as mentioned above, and discussion of unnecessary details will be hereby omitted.

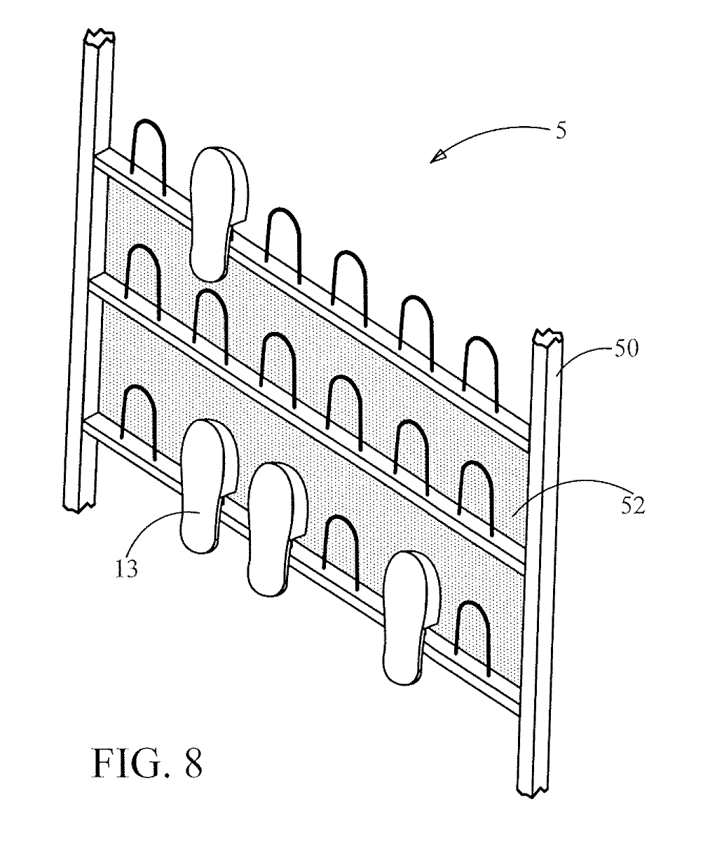

Please refer to FIG. 8, which illustrates the structure of a sterilization apparatus with a sterilization member of an embodiment of the invention. The sterilization apparatus 5 of the embodiment includes a hanging frame 50 for hanging shoes 13. Moreover, in the embodiment, the sterilization apparatus 5 further includes a sterilization member 52, such as light sterilization type member. In practice, the sterilization apparatus 5 can further includes the above-mentioned exsiccating member and ventilating member. The functionalities and types of said members are as mentioned above, and discussion of unnecessary details will be hereby omitted.

Although the present invention has been illustrated and described with reference to the preferred embodiment thereof, it should be understood that it is in no way limited to the details of such embodiment but is capable of numerous modifications within the scope of the appended claims.

What is claimed is:

1. A sterilization apparatus for a fabric and shoes comprising:
an organization member selected from the group consisting of a wardrobe, a coat hanger and a shoes cabinet; and
a light sterilization type member disposed with the organization member.

2. The sterilization apparatus of claim 1, wherein the fabric is a weave product or a non-weave product.

3. The sterilization apparatus of claim 1, wherein the light sterilization type member is an ultraviolet fluorescent light.

4. The sterilization apparatus of claim 3, wherein a photocatalyst layer is coated on the surface of the ultraviolet fluorescent light.

5. The sterilization apparatus of claim 1, wherein the light sterilization type member is an ultraviolet light-emitting diode.

6. The sterilization apparatus of claim 5, wherein a photocatalyst layer is coated on the surface of the ultraviolet light-emitting diode.

7. The sterilization apparatus of claim 1, wherein the light sterilization type member is a fluorescent lamp.

8. The sterilization apparatus of claim 7, wherein a photocatalyst layer is coated on the surface of the fluorescent lamp.

9. The sterilization apparatus of claim 1, further comprising an exsiccating member disposed with the organization member.

10. The sterilization apparatus of claim 9, wherein the exsiccating member is an infrared light.

11. The sterilization apparatus of claim 9, wherein the exsiccating member is a heating member.

12. The sterilization apparatus of claim 9, wherein the exsiccating member is an electronic exsiccating member.

13. The sterilization apparatus of claim 1, further comprising a ventilating member disposed with the organization member.

14. The sterilization apparatus of claim 13, wherein the ventilating member is a fan.

15. A sterilization apparatus for a fabric and shoes comprising:
an organization member selected from the group consisting of a wardrobe, a coat hanger and a shoes cabinet; and
an exsiccating member disposed with the organization member.

16. The sterilization apparatus of claim 15, wherein the exsiccating member is an infrared light.

17. The sterilization apparatus of claim 15, wherein the exsiccating member is a heating member.

18. The sterilization apparatus of claim 15, wherein the exsiccating member is an electronic exsiccating member.

19. The sterilization apparatus of claim 15, wherein the fabric is a weave product or a non-weave product.

20. The sterilization apparatus of claim 15, further comprising a light sterilization type member disposed with the organization member.

21. The sterilization apparatus of claim 20, wherein the light sterilization type member is an ultraviolet fluorescent light.

22. The sterilization apparatus of claim 21, wherein a photocatalyst layer is coated on the surface of the ultraviolet fluorescent light.

23. The sterilization apparatus of claim 20, wherein the light sterilization type member is an ultraviolet light-emitting diode.

24. The sterilization apparatus of claim 23, wherein a photocatalyst layer is coated on the surface of the ultraviolet light-emitting diode.

25. The sterilization apparatus of claim 20, wherein the light sterilization type member is a fluorescent lamp.

26. The sterilization apparatus of claim 25, wherein a photocatalyst layer is coated on the surface of the fluorescent lamp.

27. The sterilization apparatus of claim 15, further comprising a ventilating member disposed with the organization member.

28. The sterilization apparatus of claim 27, wherein the ventilating member is a fan.
